(19) United States
(12) Patent Application Publication
(10) Pub. No.: US 2007/0129777 A1
(43) Pub. Date: Jun. 7, 2007

(54) Light therapy device

(76) Inventor: Charles Bolta, Boulder, CO (US)

Correspondence Address: EMERY L. TRACY, P.O. BOX 1518, BOULDER, CO, 80306, US

(21) Appl. No.: 11/602,928

(22) Filed: Nov. 21, 2006

Related U.S. Application Data
(60) Provisional application No. 60/739,979, filed on Nov. 23, 2005.

(51) Int. Cl. A61N 5/06

(52) U.S. Cl. 607/88

(57) ABSTRACT

A light therapy device for alleviating medical disorders is provided. The light therapy device comprises a housing having a pair of first side walls, a pair of second side walls, a pair of side walls, and a bottom wall with the second side walls being between the first side walls and the bottom wall, the first side walls being substantially perpendicular to the bottom wall, and the side walls being substantially perpendicular to the first side walls and the bottom wall. A light bulb is positioned within the housing. An angled light reflective panel is positioned between the bottom wall of the housing and the light bulb wherein the light reflective panel reflects light and heat in a general direction toward the first side walls, the second side walls, and opposite the bottom wall thereby removing light and heat from behind the light bulb.

The present application claims benefit of priority of pending provisional patent application Ser. No. 60/739,979, filed on Nov. 23, 2005, entitled “10,000 Lux Mini”.

### BACKGROUND OF THE INVENTION

1. Field of the Invention

The present invention relates generally to a light therapy device for alleviating the specific medical disorder known as Seasonal Pattern or Seasonal Affective Disorder, also know as SAD or the winter blues, based on the Theory of Light, which recommends 10,000 lux and the highest spectrum rated lux bulbs and, more particularly, the invention relates to a light therapy device having efficient placement and effective utilization of a particular treatment modality in an institutional, home or business setting and can also be used to illuminate a room or used as a shop or detailed work light.

2. Description of the Prior Art

Little information is presently available with respect to treating Seasonal Pattern or Seasonal Affect Disorder. Basically, the disorder appears treatable to some extent by subjecting the patient to natural sunlight, or a full-spectrum light; that is a light which emits throughout the visible spectrum at wavelengths approaching the spectrum at which the sun's light reaches the surface of the earth; that is the wavelengths to which man has been subjected throughout evolution. While treating the disorder appears simple enough in actual practice, problems of both a mechanical and electromagnetic nature have not as yet been appropriately addressed.

Considering the nature and effectiveness of the usage, the question arises as to how to properly shield the electric fields and radio frequency fields emanating from the lights. It has been determined that there is potentially compelling scientific evidence that suggests that exposure to electric fields and radio frequency fields may not only generally cause physical and emotional disorders in human beings, but that these impact might negatively impact the specific treatment being applied in this case. Thus, it is preferred that a system must act to reduce these fields as much as possible.

In addition, any device used for treatment of Seasonal Pattern must be sturdy, strong, movable, and easily cleansed while requiring low maintenance. Other factors taken into consideration in designing a device is that it be easily repaired and cleaned while being steady during use.

Non-Pharmacological Solution

While information appears to be limited on non-pharmacological ways to combat the winter blues, also known as Seasonal Affective Disorder, there is a need to provide a device that is not a drug, thus avoiding pharmacological contraindications or issues.

Compliance

Compliance is an important factor in any therapy approach, therefore having a lightweight, portable, effective and affordable light therapy device available is necessary. Also, the device must be portable for travel.

Electric and Radio Frequency Fields

Another consideration is providing a light therapy device that properly shields the electric fields and radio frequency fields that emanate from the light and light fixture. There are scientific studies that indicate exposure to electric fields and radio frequency fields may not only generally cause both physical and emotional disorders in human beings, but that these impacts may negatively impact the specific treatment being implemented in this case. Therefore, it is favored to use a light therapy device that substantially reduces electric fields and radio frequency fields as much as possible.

Solving the Problems

In order to solve the aforementioned problems (providing a non-drug solution: providing a lightweight, portable, effective and affordable solution; minimizing electric and radio frequency fields) it is important to develop a lightweight, compact, low electric field and low radio frequency field light therapy device that can handle the new generation advanced technology FML fluorescent light bulbs and effectively and safely maximize the light output.

To solve the mechanical problems at hand, a light therapy device is needed that can be quickly and efficiently positioned, allowing simple operation, and abundantly illuminating the area where such persons using it are seated.

### SUMMARY

It is an object of the present invention to provide a light therapy device which is portable, easily assembled, and having an adjustable base foot plate to position the light therapy device in multiple positions containing full-spectrum, high lumen output bulbs, while also providing shielding from measurable electric and radio frequencies.

Another object of the present invention is to provide a full-spectrum light therapy device having light that is the closest to natural sunlight available. The light therapy device should also provide very bright lumen, lux, or foot-candle output.

Still another object of the present invention is to provide a light therapy device that shields the patient from the potentially harmful effects of electric and radio frequency field's emissions. Included in the goal of shielding is reducing the measurable AC electric field and radio frequencies through the front prismatic lens with a wire grid or a ¾×¾×¾ open cube metal or metal coated plastic lens, with minimal loss of light and without altering the full-spectrum or blue spectrum light that passes through it.

Yet another object of the present invention is to provide a light therapy device reflecting light from behind a flat compact FML type bulb. The multi-angled three stage reflective heat convection channel reflects light through the bulbs, from in back of the bulb to angled sidewalls, and out the lens face while reducing heat in the fixture.

The light therapy device is a lightweight, compact, portable aluminum fixture with venting for maximizing convection heat dissipation to meet U.L. standards for heat. The light therapy device is constructed to maximize light output and reduce heat inside of the fixture by using a multi-angled light reflective heat convection channel with the compact fluorescent light bulb known as a FML bulb. The light therapy device also minimizes electric frequency and radio frequency fields and can be powder coated to reduce off gassing.

Each light therapy device includes the fixture having a flicker-free electronic ballast that is more energy efficient and reduces magnetic fields, internal radio frequency filter for grounding out unnecessary radio frequency fields which can interfere with radio and television reception, an on-off switch, three-prong grounded cord, a FML55 watt full spectrum (93 C.R.I., 5550° Kelvin) 10,000 lux bulb, a spectrally transparent prismatic wire inlayed plastic filter lens that minimally alters the quality of light and will not yellow with age. Or a ¾×¾×¾ open cube metal or metal coated plastic lens, with minimal loss of light output and without altering the full-spectrum or blue spectrum light that passes through it. The cube size of lens can vary between ¾″ to 6″ and we have found that the ¾″ by ¾″ by ¾″ cube lens had the best results for reducing electric and radio frequencies and also allowing the light to pass through. The lens are grounded to the body of fixture and are U.L. approved.

Furthermore, the light therapy device is easy to operate, affordable, and aesthetically pleasing for consumers.

### BRIEF DESCRIPTION OF THE DRAWINGS

Before explaining the disclosed embodiment of the present invention in detail, it is to be understood that the invention is not limited to its application to the details of the particular arrangement as shown, since the invention is capable of different embodiments. Also, the terminology used herein is for the purpose of description and not of limitation.

FIG. 1 is a perspective view illustrating a light therapy device, constructed in accordance with the present invention, with a lens in place and a base foot plate maintaining the light therapy device on a flat surface;

FIG. 2 is a perspective view illustrating the light therapy device, constructed in accordance with the present invention, with the light therapy device having convection heat vent holes and positioned vertically on a horizontal surface;

FIG. 3 is a perspective view illustrating the light therapy device, constructed in accordance with the present invention, with horizontal positioning of the light therapy device;

FIG. 4 is a perspective view illustrating the light therapy device, constructed in accordance with the present invention, with the top end cap removed allowing the front lens to be removed;

FIG. 5 is a perspective view illustrating the light therapy device, constructed in accordance with the present invention, with the base foot plate allowing the light therapy device to be positioned on a horizontal surface;

FIG. 6 is an elevational front view illustrating the light therapy device, constructed in accordance with the present invention, with the end top cap removed and the FML bulb installed;

FIG. 7 is an end view illustrating the light therapy device, constructed in accordance with the present invention, with a three-stage reflective panel and heat channel positioned behind the FML bulb reflecting the light from behind the bulb and out through the lens; and

FIG. 8 is a front view illustrating the light therapy device, constructed in accordance with the present invention, with a wire grid, lens cover slidable into the fixture through a lens slide track channel, and lens electrical ground contact points on the lens.

### DETAILED DESCRIPTION OF THE PREFERRED EMBODIMENTS

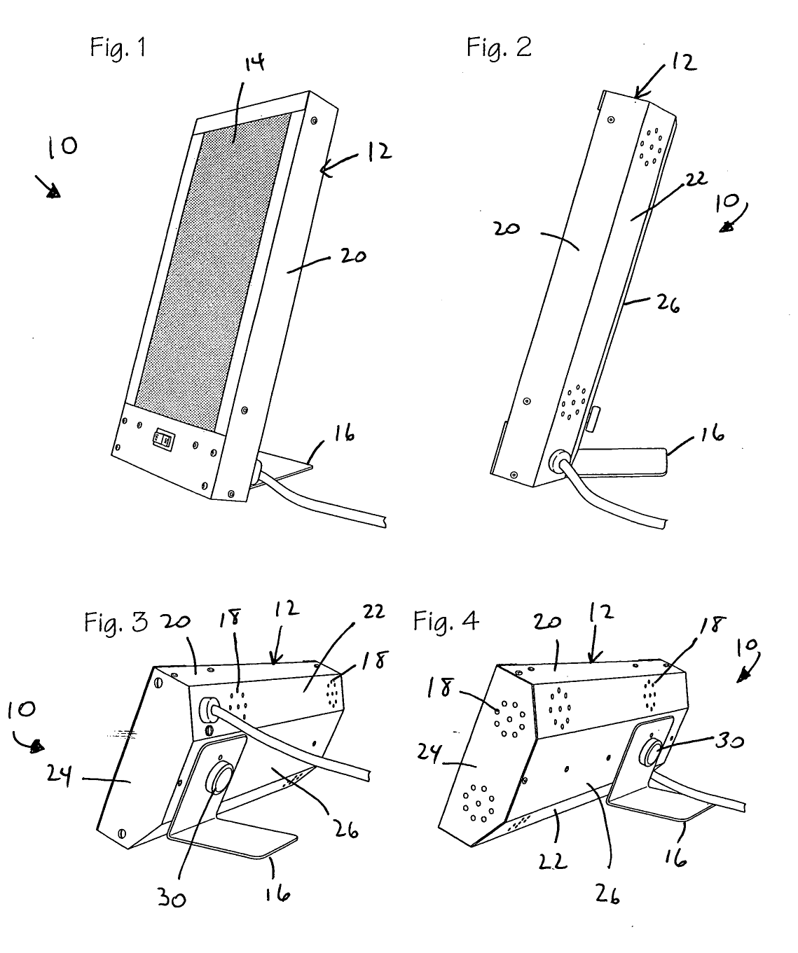

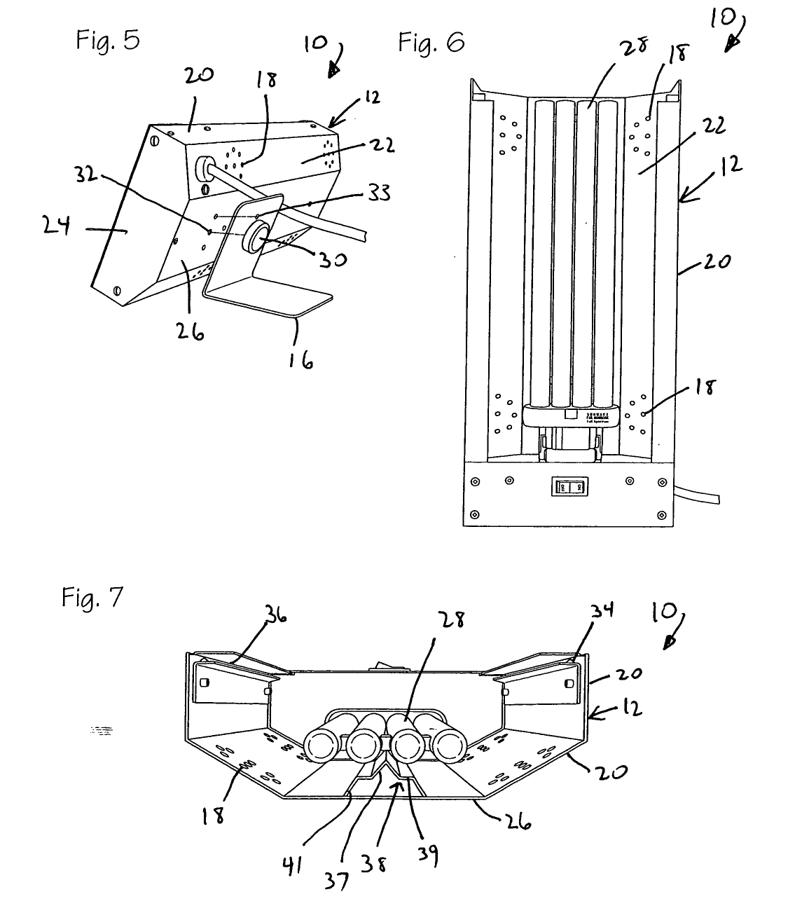

As illustrated in FIGS. 1-8, the present invention is a light therapy device, indicated generally at 10, for alleviating the specific medical disorder known as Seasonal Pattern or Seasonal Affective Disorder, also know as SAD or the winter blues, based on the Theory of Light, which recommends 10,000 lux and the highest spectrum rated lux bulbs.

As illustrated in FIGS. 1 and 2, the light therapy device 10 of the present invention includes a fixture or housing 12 having a pair of first side walls 20, a pair of second side walls 22, a pair of end walls 24, and a bottom wall 26. The second side walls 22 are angled approximately eighteen (18°) degrees between the first side walls 20 and the bottom wall 26 (measured from horizontal), the first side walls 20 are substantially perpendicular to the bottom wall 26, and the end walls 24 are substantially perpendicular to the first side walls 20 and the bottom wall 26. The angle of the second side walls 22 presented herein is a preferred embodiment and other angles of the second side walls 22 are within the scope of the present invention.

The light therapy device 10 of the present invention further includes a lens 14 securable to the fixture 12 and a base foot plate 16 releasably secured to the fixture 12 for maintaining the fixture 12 on a horizontal surface (not shown). The light therapy device 10 further includes convection heat vent holes 18 to convect heat through either side of the fixture 12.

As illustrated in FIGS. 3-6, the light therapy device 10 of the present invention is horizontally positionable on a horizontal surface with the heat vent holes 18 allowing the heat to convect either through the side walls 20, 22, the end walls 24, and/or the bottom wall 26. The number of vent holes 18 can have an artistic design for retail sales and either meet or exceed U.L. requirements for heat dispersion. The hole patterns and number of holes 18 can be increased or decreased depending on the heat output of the bulbs used.

As noted, the light therapy device 10 can be positioned either horizontally or vertically and convect heat either horizontally, vertically, side to side, or bottom to top allowing the light therapy device 10 to be cooled without the added expense of a fan. The horizontal position of the light therapy device 10 allows the light therapy device 10 to be adjusted between approximately thirty-five (35°) degrees and approximately one hundred and eighty (180°) degrees to bath a user's face thereby maximizing exposure to the light, as needed. The angled sides 20, 22 of the fixture 12 allow the light therapy device 10 to be positioned on a table without falling over. The foot base plate 16 can be bent at a bend line allowing the user to adjust and/or change the angle to their personal preference. The base foot plate 16 can be angled by bending the foot plate 16 from approximately one hundred and eighty (180°) degrees to approximately thirty-five (35°) degrees allowing a person to be bathed by different types of angled light effects.

The end walls 24 of the fixture 12 of the light therapy device 10 of the present are preferably removable. The end walls provide structural strength to the fixture 12 by holding the light therapy device 10 together, allowing the front lens 14 to be easily removed, and holding the lens 14 in place. The end walls 24 also have the heat releasing vent holes 18 allowing the heat from the compact fluorescent bulb, i.e., the FML bulb 28, which is flat, very hot, and very compact. The end wall holes 18 can be positioned to form an aesthetically pleasing design as compared to industrial slits. The hole patterns are an intentionally a round, circular design for a better appeal to the retail public. In addition, the end walls 24 pass U.L. requirements for heat temperature on the surface. The hole patterns can be increased or decreased depending on heat output of any bulbs 28 used.

The base foot plate 16 of the light therapy device 10 of the present invention also spins and adjusts on a set screw handle 30. Punched holes 32 in the fixture 12 allow the light therapy device 10 to be positioned at predetermined locations. The base foot plate 16 also has a nub 33 receivable within one of the punch holes 32 in the fixture 12 and securing means extending through the base foot plate 16 into another one of the punch holes 32 in the fixture 12. The nub 33 maintains the base foot plate 16 from rotating relative to the fixture 12. The base foot plate 16 can also be adjusted to any angle desired other than the predetermined punch set up on the base foot plate 16 and the fixture 12 allowing a user alternative angles of light distribution having more effect on the light entering the eye rather than the fixture 12 just sitting at approximately ninety (90°) degrees on a horizontal surface.

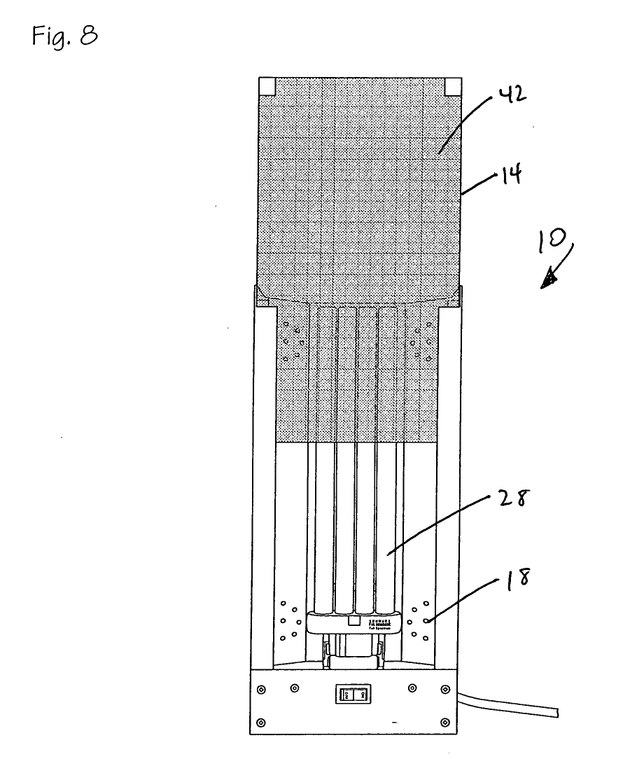

As illustrated in FIG. 7, the light therapy device 10 of the present invention has an electrical ground contact point 34 on a lens slide rack 36 formed in the first side walls 20. The lens 14 slides easily along the lens slide track 36 to remove the bulb 28 from or add a bulb 28 to the light therapy device 10. Removing the lens 14 from the front slide design allows for easy and effortless removal. The lens slide rack 36 also has the contact point 34 at the top of the fixture 12 in addition to another contact point 34 anywhere along the lens slide rack 36 allowing contact for a lens 14 having a metallic contact point connected to a wire grid in the plastic lens 14 or has a metallic coating on a parabolic lens. The contact point 34 allows the lens 14 to be grounded through the fixture 12 to a three-prong grounded electrical cord system. The grounding allows the light therapy device 10 to effectively reduce the electric fields and radio frequencies that are emitted through a fluorescent bulb and fixture. Since the fields have a measurable number and with a grounded wire grid lens or with a parabolic lens with metallic coating, the contact point 34 allows those fields to be reduced. The contact point 34 allows sliding of the lens 14 through the fixture 12 without having to hardwire the lens 14 to the fixture 12 which makes the unit unsafe and difficult for the user to install the lens cover.

In addition, the light therapy device 10 of the present invention includes a three-stage reflective panel 38 with a heat channel 40 positioned between the FML bulb 28 and the bottom wall 26 to reflect the light from behind the bulb 28 and out through the lens 14. The reflective panel 38 positioned behind the bulb 28 can be constructed with either single, dual, or three-stage reflectors reflecting light off of the angle of the reflective panel 38 to the side walls 20, 22 and then out the lens 14. The reflective panel 38 also bounces the light up inside the fixture and removes light from behind the bulb 28.

The reflective panel 38 has three surfaces for reflecting light and heat. The first surface 37 has an angle of approximately fifty (50°) degrees, the second surface 39 has an angle of approximately one hundred and eighty (180°) degrees, and the third surface 41 has an angle of approximately sixty-five (65°) degrees. All angles are measured from horizontal. The three surfaces bounce the light against the first and second side walls 20, 22 and through the bulb 28. It should be noted that the angles of the three surfaces presented herein are only a preferred embodiment and other angles for the three surfaces are within the scope of the present invention.

Preferably, the reflective panel 38 is constructed from a metal material acting as a heat sync shield and keeping the heat from emanating from the bulb 28 and going to the fixture 12. The heat channel 40 of the reflective panel 38 draws heat from the channel up to the end wall 24 and out of the fixture 12. The actual convection can occur from the open bottom of the heat channel 40 and the side vent holes 18 of the light therapy device 10 and convect up through to the end walls 26. Preferably, the end of the heat channel 40 adjacent the fixture 12 of the light therapy device 10 is open and not completely closed allowing the heat convection to pass through the heat channel 40 thereby allowing the light therapy device 10 to operate as a single, dual, three-stage reflector. The light therapy device 10 can also operate as a heat sync allowing heat convection to draw up toward the end walls 26 with the light refracting off the side angles of the reflective panel 38 to the side walls 20, 22 and reflecting light up and out with light scattering up through the lens 14 area or through the spaces between the bulb 28.

The reflective panel 38 can either be a single unit behind the bulb 28 or multiple reflective panels can be positioned behind the bulbs 28 reflecting light out and away from the end walls 26 of the light therapy device 10. The reflective panel 38 can also be a single reflective plane movable between any of the bulbs 28 and reflecting light one direction without being connected to each other.

As illustrated in FIG. 8, the light therapy device 10 of the present invention further includes a wire grid 42 covering the lens 14 sliding into the fixture 12 through the lens slide rack channel 36, and the lens electrical ground contact points 34 on the lens 14.

While the light therapy device 10 of the present invention has been designed using the new generation, high output FML compact fluorescent bulb and to maximize the new technology in a marketplace that continually seeks and embraces compact technology while solving the problems which may arise from the use of this new technology, the light therapy device 10 can be used with any type of bulb 28. The new generation FML bulb is a compact bulb emitting a very bright light and producing higher heat levels. One if its attributes is that is the bulb is flat in nature blocking its own light from reflecting from the back of the bulb and off of the back of the light therapy device 10. Therefore, light emanates primarily from the front of the bulb through the lens area so only about fifty (50%) percent of the light is available.

To solve for the refracting issue, the light therapy device 10 of the present invention uses specialized heat shielding, channeling, and a heat venting multi-reflective lens behind the bulb to harness the light bouncing from the bottom wall 26 of the fixture 12 and the bulb 28 and out the front lens 14.

To solve the problem of heat produced by the FML bulb, the light therapy device 10 has a two way approach. First, the light therapy device 10 is preferably constructed from aluminum for dissipating the heat to the outside side walls 20, 22. Second, the light therapy device 10 has venting holes 18 throughout. The venting holes 18 coupled with the use of an aluminum body dissipate the heat and meets or exceeds U.L. requirements. The novel multi-reflective three-stage reflective panel 38 dissipates heat from the bottom wall 26 of the fixture 12 and behind the bulb 28 thereby channeling the heat out and through the vent holes 18. Heat vent holes 18 can also be formed in the bottom wall 26 underneath the heat channel 40 to vent the heat out or convect the heat out through the end walls 24.

The light therapy device 10 of the present invention is a very efficient, lightweight travel bulb light box for winter depressions, circadian cycle shifts, and jet lag. A user can easily move the light therapy device 10 when they travel or in any environment, i.e., in the office or home. Conventional light boxes are not able to handle the heat issues and the bulbs are very bright. The light therapy device 10 facilitates heat reduction. The fixture 12 can be set up three (3) different directions—it can set vertically or horizontally on its right or left side and heat dissipation or convection of the heat through the fixture 12 is designed specifically to allow that heat to dissipate, in all three (3) directions and still meet U.L. heat specifications, without the added expense of a cooling fan. The bulb 28 for the lighting therapy device 10 is preferably a FML type that is flat and blocks its own light from reflecting from the back and sides of the bulb 28 with the sides of the reflective panel 38 having reflective angles channeling light bouncing from the back angled heat shield channel reflector to the side walls then out the front lens cover. The angled side panels can range from approximately fifteen (15°) degrees to approximately forty-five (45°) degrees or more to approximately ninety (90°) degrees. The multi-reflective heat channel can range from approximately ninety (90°) degrees to approximately one hundred and forty (140°) degrees or more. The multi-reflective heat channel can also be just one angle between approximately ninety (90°) degrees and approximately one hundred and forty (140°) degrees “V” shaped. The angles for both multi and single reflection heat channel can be an inverted “V” shape to approximately ninety (90°) degrees or forty-five (45°) degrees or more.

Once turned on, the light therapy device 10 automatically produces the predetermined amount of high lumen and/or full spectrum light output and the electric and radio frequency fields are reduced as a result of the design construction noted previously. In addition to the light therapy device 10 being used for light therapy, the light therapy device 10 can be utilized in alternative settings such as a portable shop light, task light, or artist's light providing bright light to a task or showcase an object.

The inclusion of a ¾″ by ¾″ by ¾″ chrome plated parabolic cube lens 14 allows for the electric and radio frequency fields to be reduced from the front of the light therapy device 10 with the least amount of light distortion. The prismatic lens 14 has a wire grid 42 encased within the plastic lens 14. The cube parabolic lens with chrome plating is grounded to the metal frame of the light box with a contact point 34 in the lens' slide rack 36. The grounding lowers the amount of measurable electric and radio frequency fields being emitted by the light therapy device 10 when turned on (as measured by the amount of 60 Hz AC voltage detectable near the surface of the lens at distance of up to one meter). Grounding is important since certain emissions can actually match, or at least impact, low-frequency brainwave and other biological patterns.

In spite of the fact that the light can often operate at a household frequency (60 Hz), it appears that varying frequency electric and radio frequency fields are possible. The grounding feature on the lens structure can act as an RF filter or to otherwise serve to mitigate the effect of electric and radio frequency fields. Grounding can also act to avoid allowing an impact on the patient regardless of the exact nature of the effect. The parabolic lens 14 can also be used to maximize the amount of full-spectrum light able to come through without altering the light. It has been found that with a larger eighteen cell parabolic lens in a four (4′) feet by two (2′) feet fixture 12, the measurable electric and radio frequency fields are not sufficiently removed. The use of a one-inch square mesh wire grid 42 attached parallel to a prismatic lens 14 lowers the measurable electric and radio frequency fields to a satisfactory level and also allows for a maximum amount of full-spectrum light to come through the lens 14.

The following are advantages and benefits of the present invention:
1. Very tight, compact, winter depression lighting unit with angles to reflect light out.
2. A base foot plate providing the ability for the light therapy device 10 to be kept upright or angled backwards to shine upwards or on one side, right side or left side. The unit can be vertical or horizontal to the right or horizontal to the left. The base foot plate can be moved with set screw to three different directions with a tightening motion with an indentation in the box and also an indentation that fits into the indentation on the box on the base foot plate. The base foot plate has a punch out that fits into a punch out that is on the box also.
3. Heat dissipation through venting at the bottom and also at the top end caps. The heat dissipation of a specific amount to meet U.L. requirements/approval for heat dissipation of a closed unit. The way the heat dissipation has been designed it can disperse heat from when it sits vertically on the table and also when it sits horizontally either from the right side or the left side, without the added expense of a cooling fan.
4. Minimizing electromagnetic fields and radio frequencies. The face plate can be removed by sliding it through a channel that allows the lens to slide in and out of the unit. The top cap can also be removed. The prismatic lens also can have a wire grid in it with contact points which can touch the sliding rail that it slides into. At one end, the sliding rail is crimped and the lens slides in. The contact for the lens grid removes electric fields and radio frequencies by making contact with the sliding channel and completing the grounding circuit. The contact points are formed from crimping of the sliding channel at the top end to allow contact to take place.
5. The cap on the top has the venting holes, also dual purpose, and a cap for the unit and also a venting system.
6. The lens cover for the fixture can either be a plastic prismatic lens with wires grid with contact points or a cube lens allowing light through it. It has been found that ¾×¾×¾ inch metal or plastic cube with chrome or other metal platings are able to reduce the electric fields and the radio frequencies off the unit and make a measurable difference. The channel can be adjusted for holding either the cube lens or the prismatic lens.
7. The angling of the actual box will reflect light from the box.
8. A chrome reflective panel in the back of the unit like a pyramid from the frame out refracts the light in the back side to the side walls and out through the front of the lens therefore maximizing the amount of light that the light therapy device 10 emits.

Each unit comes complete with an on/off switch and a standard three-prong plug. Once turned on, the unit automatically produces the predetermined amount of high lumen or full-spectrum light, and the electric and radio frequency field's emissions are reduced through proper design as discussed earlier.

The light therapy device 10 of the present invention provides a person dealing with Seasonal Affective Disorder or the winter blues the closest to natural sunlight possible while simultaneously mitigating the potentially harmful effects of electric and radio frequency field's emissions. The mitigation of electric and radio frequency fields takes into consideration that a person with SAD may already be in a deteriorated physical and/or emotional state, and therefore more susceptible to electric and radio frequency fields. It is also preferred that the light therapy device can be assembled and used by one person.

Conclusion

In general, it is an object of the present invention to provide a compact, portable, affordable, aesthetically pleasing, affordable light therapy unit that contains full spectrum high lux light bulb while providing shielding from measurable electric fields and radio frequencies.

In sum, the present invention is a lightweight portable lighting unit which produces high light output in a small compact fixture for humans or animals that are in need of the exposure to intense light which is known as light therapy. This unit produces intense full spectrum white light incorporating 10,000 lux output, and is extremely compact in size for the light output. The unit is light weight, has an aluminum body is vented from multiple directions so the unit can sit on a flat surface in multiple directions, i.e. vertical, horizontal, either right or left horizontal. The unit has a heat dissipation channel with reflective properties, behind the bulb, and within the unit body walls to allow as much light and heat to exit the unit using new FML design compact fluorescent bulbs. The unit employs the design of minimizing electric fields and radio frequency fields.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the present invention is to be limited only to the claims except as precluded by the prior art. Moreover, the invention as disclosed herein, may be suitably practiced in the absence of the specific elements which are disclosed herein.

What is claimed is:

1. A light therapy device for alleviating medical disorders, the light therapy device comprising:
a housing having a pair of first side walls, a pair of second side walls, a pair of end walls, and a bottom wall, the second side walls being between the first side walls and the bottom wall, the first side walls being substantially perpendicular to the bottom wall, the end walls being substantially perpendicular to the first side walls and the bottom wall;
a light bulb positioned within the housing; and
an angled light reflective panel positioned between the bottom wall of the housing and the light bulb;
wherein the light reflective panel reflects light and heat in a general direction toward the first side walls, the second side walls, and opposite the bottom wall thereby removing light and heat from behind the light bulb.

2. The light therapy device of claim 1 and further comprising:
a heat channel formed between the angled light reflective panel and the bottom wall.

3. The light therapy device of claim 1 and further comprising:
a plurality of apertures formed in a wall selected from the group consisting of the first side walls, the second side walls, the end walls and the bottom wall;
wherein heat generated by the light bulb dissipates through the holes.

4. The light therapy device of claim 1 and further comprising:
a grounded ¾×¾×¾ open cube metal or metal coated plastic lens, the cube size of the lens varying between approximately ¼″ to 6″.

5. The light therapy device of claim 1 wherein the housing is constructed from an aluminum material meeting U.L. requirements for reducing heat.

6. The light therapy device of claim 1 and further comprising:
a removable foot plate securable to the side walls or the bottom wall of the housing, the foot plate having a bend line allowing adjustment of the light therapy device to a desired angle.

7. The light therapy device of claim 6 wherein the foot plate can be angled by from approximately thirty-five (35°) degrees to approximately one hundred and eighty (180°) degrees.

8. The light therapy device of claim 6 wherein the foot plate is rotatable and adjustable by a set screw handle, the foot plate having a nub receivable within a punch hole in the housing and securing means extending through the foot plate into another punch hole in the housing.

9. The light therapy device of claim 1 wherein the light reflective panel has a pair of first angled sides, a pair of second angled sides, and a pair of third angled sides, the first pair of angle sides reflecting light and heat in a general direction toward the first side walls of the housing, the second pair of angled sides reflecting light and heat in a general direction opposite the bottom wall of the housing and through spacing between individual light bulbs, and the third angled sides reflecting light and heat in a general direction toward the second side walls.

10. The light therapy device of claim 1 and further comprising:
a lens slide track formed in the first side walls of the housing; and
a lens slidable into the lens slide track.

11. The light therapy device of claim 10 wherein the lens has a wire grid or metallic coating and further comprising:
an electrical ground contact point formed on the lens slide track.

12. A light therapy device for alleviating medical disorders, the light therapy device comprising:
a housing having a pair of first side walls, a pair of second side walls, a pair of end walls, and a bottom wall, the second side walls being between the first side walls and the bottom wall, the first side walls being substantially perpendicular to the bottom wall, the end walls being substantially perpendicular to the first side walls and the bottom side wall;
a light bulb positioned within the housing;
an angled light reflective panel positioned between the bottom wall of the housing and the light bulb;
a heat channel formed between the angled light reflective panel and the bottom wall;
a plurality of apertures formed in a wall selected from the group consisting of the first side walls, the second side walls, the end walls and the bottom wall;
a removable foot plate securable to the side walls or the bottom wall of the housing, the foot plate having a bend line allowing adjustment of the light therapy device to a desired angle;
a lens slide track formed in the first side walls of the housing; and
an open cube lens slidable into the lens slide track;
wherein the light reflective panel reflects light and heat in a general direction toward the first side walls, the second side walls, and opposite the bottom wall thereby removing light and heat from behind the light bulb; and
wherein heat generated by the light bulb dissipates through the holes.

13. The light therapy device of claim 12 wherein the foot plate can be angled by from approximately thirty-five (35°) degrees to approximately one hundred and eighty (180°) degrees.

14. The light therapy device of claim 12 wherein the foot plate is rotatable and adjustable by a set screw handle, the foot plate having a nub receivable within a punch hole in the housing and securing means extending through the foot plate into another punch hole in the housing.

15. The light therapy device of claim 12 wherein the light reflective panel has a pair of first angled sides, a pair of second angled sides, and a pair of third angled sides, the first pair of angle sides reflecting light and heat in a general direction toward the first side walls of the housing, the second pair of angled sides reflecting light and heat in a general direction opposite the bottom wall of the housing and through spacing between individual light bulbs, and the third angled sides reflecting light and heat in a general direction toward the second side walls.

16. The light therapy device of claim 12 wherein the lens has a wire grid or metallic coating and further comprising:
an electrical ground contact point formed on the lens slide track.

17. A method for alleviating medical disorders, the light therapy device comprising:
providing a housing having a pair of first side walls, a pair of second side walls, a pair of end walls, and a bottom wall;
positioning the second side walls between the first side walls and the bottom wall;
positioning the first side walls substantially perpendicular to the bottom wall;
positioning the end walls substantially perpendicular to the first side walls and the bottom wall;
mounting a light bulb within the housing;
mounting an angled light reflective panel between the bottom wall of the housing and the light bulb; and
reflecting light and heat in a general direction toward the first side walls, the second side walls, and opposite the bottom wall thereby removing light and heat from behind the light bulb.

18. The method of claim 17 and further comprising:
forming a heat channel between the angled light reflective panel and the bottom wall.

19. The method of claim 17 and further comprising:
forming a plurality of apertures in a wall selected from the group consisting of the first side walls, the second side walls, the end walls and the bottom wall; and
dissipating heat generated by the light bulb through the holes.

20. The method of claim 17 and further comprising:
forming a lens slide track in the first side walls of the housing; and
sliding a lens into the lens slide track.
